(19) United States
(12) Patent Application Publication
(10) Pub. No.: US 2016/0376694 A1
(43) Pub. Date: Dec. 29, 2016

(54) Method and Apparatus for Coating Nanoparticulate Films on Complex Substrates

(71) Applicant: ECOLE POLYTECHNIQUE FEDERALE DE LAUSANNE (EPFL), Lausanne (CH)

(72) Inventors: Juan Kiwi, Préverenges (CH); Sami Rtimi, Préverenges (CH); César Pulgarin, Lausanne (CH)

(21) Appl. No.: 15/039,457

(22) PCT Filed: Nov. 27, 2013
(86) PCT No.: PCT/IB2013/060439

§ 371 Date: May 26, 2016

(51) Int. Cl. C23C 14/06; C23C 14/35; C23C 14/54; H01J 37/34; A01N 59/16; A61L 29/10; A61L 31/16; A61L 31/08; A01N 59/20; C23C 14/34; A61L 29/16

(52) U.S. Cl. CPC C23C 14/0688; C23C 14/3414; C23C 14/3485; C23C 14/35; C23C 14/54; H01J 37/3405; H01J 37/3429; H01J 37/3467; H01J 37/3476; A61L 29/16; A61L 29/106; A61L 31/16; A61L 31/088; A01N 59/20; A01N 59/16; A61L 2300/404

(57) ABSTRACT

Active films and processes for depositing the same onto a complex 3D shape substrates and implants are provided. The process comprises the following steps: inserting into a process chamber a sputtering target, including at least two chemical elements and a complex shape 3D substrate on a substrate holder, providing a gas to be ionized into the process chamber with a controlled pressure; applying a voltage in pulse between the sputtering target and the complex shape 3D substrate; and generating a magnetic field at the surface of the sputtering target inside the process chamber as required for HIPIMS.

### TECHNICAL FIELD

This invention relates to a method for forming active nanoparticulate films on complex shape 3D surfaces, catheters and implants. In a particular aspect, the active film is coated directly on fabrics or on threads and presents a fast antimicrobial effect.

### BACKGROUND

There is a constantly increasing demand for the development of tailor-made films with highly specific features: hardness, wear or corrosion resistance, low friction, specific electrical, optical or chemical behaviour, porosity. The increasing requirements on films imply the need for developing new advanced film processes.

The need for effective active surfaces is well established, specifically for antimicrobial surfaces in various environments including hospitals, industry and even home. Medical devices, linens and clothing among other can provide a suitable environment for many bacteria, fungi or viruses to grow which allows the transmission of infectious diseases.

There are various ways to manufacture active thin films; electroplating, chemical vapour deposition (CVD), evaporation (laser, plasma assisted . . . ) as well as combinations of those methods. Most of these methods may have drawbacks, among other the difficulty to control the homogeneity of the nanoparticulate films.

Although an example of Cu/TiO₂ anti-microbial films is to be discussed as preferred embodiment hereafter, it is to be understood that the same augments would apply for other active particles or nanoparticles embedded in a matrix in order to provide an active film.

Antimicrobial surfaces can reduce/eliminate hospital-acquired infections (HAI) acquired on contact with bacteria surviving for long times in hospital facilities [1-2]. To preclude/decrease viral, nosocomial infections and antibiotic resistant bacteria Borkow and Gabbay [3] introduced Cu into textile fabrics. Recently Sunada et al., [4-5] and Torres et al., [6a] and O. Akhavan [6b-6d] have recently reported the preparation of the Cu and TiO₂/Cu films by sol-gel methods with materials absorbing in the visible range.

These sol-gel deposited films are not mechanically stable. In many cases their preparation is not reproducible and does not present uniformity but only low adhesion since they can be wiped off by a cloth or thumb [7]. Additionally, the substrate needs to be pre-treated in order to allow the sol-gel film to be stabilized onto the substrate surface. This is an expensive, time consuming and energy intensive step. The sol-gel based films are highly inhomogeneous specifically when applied on complex shapes devices. Additionally, the thickness of the sol-gel films has a significant impact on the texture of the textile on which the film is coated.

In recent years physical vapor deposition (PVD) has been used to produce antimicrobial films by condensation of a vaporized precursor onto the substrate at relatively high temperatures. Page et al., [8], Foster et al., [9], Dunlop et al., [10] and Page et al., [11] have reported antibacterial films preparation of Ag and Cu on glass and thin polymer films by PVD. TiO₂, Ag, and Cu films 6 to 50 nm thick have been shown to inactivate bacteria under UV and in some cases under visible light irradiation. The disadvantages of the CVD deposition approach are the high investment costs, the high temperatures needed precluding film deposition on textiles besides the large amount of heat used requiring costly cooling systems. Additionally a pre-treatment of the surface is often needed and the process temperature is not adapted to all substrates. Even if the thickness of the obtained film is smaller than the ones obtained through the sol-gel processing, it has still a significant impact on the texture of the coated substrate.

High power impulse magnetron sputtering (HIPIMS) has been used recently to prepare films by applying strong power pulses leading to sputter layers presenting high adherence, complete coverage and superior resistance against corrosion and oxidation [12-13]. One of the main problems encountered when depositing uniform Cu-films by direct current pulsed magnetron sputtering (DC/DCP) [13] is that deposition on rough and complex shape substrates is not uniform.

### SUMMARY OF THE INVENTION

In a first aspect, the invention provides a process for depositing a film onto a complex 3D substrate which comprises the following steps: inserting into a process chamber a sputtering target, including at least two chemical elements and a complex 3D substrate on a substrate holder, providing a gas to be ionized into the process chamber with a controlled pressure; applying a voltage in pulse between the sputtering target and the complex 3D substrate; and generating a magnetic field at the surface of the sputtering target inside the process chamber as required for HIPIMS.

In a first preferred embodiment, the at least two chemical elements are selected from the group consisting of transition metals, poor metals, metalloids or polyatomic nonmetals.

In a second preferred embodiment, the at least two chemical elements are copper (Cu) and titanium dioxide (TiO₂).

In a third preferred embodiment, the at least two different chemical elements are present in a ratio of 40 at. % for copper (Cu) and 60 at. % for titanium oxide (TiO₂).

In a fourth preferred embodiment, the process further comprises a step of controlling a distance between the sputtering target and the substrate to be coated in the process chamber.

In a fifth preferred embodiment, the distance between the sputtering target and the substrate to be coated is set at 10.5 cm.

In a seventh preferred embodiment, the gas is a mixture of an inert gas and a reactive gas.

In an eighth preferred embodiment, the gas is a mixture of Argon and Oxygen.

In a ninth preferred embodiment, the mixture of Argon and Oxygen is in a ratio of FluxO₂/FluxAr=0.05.

In an eleventh preferred embodiment, the voltage is applied so that the pulse has a power per pulse in a range of 1000 W to 2000 W and has a duration in a range of 50 μs to 200 μs.

In a twelfth preferred embodiment, the process is further characterized in that the power per pulse is 1750 W and the pulse has duration of 100 μs.

In a thirteenth preferred embodiment, the process further comprises the step of selecting process conditions as a sputtering target composition, a distance between the sputtering target and the substrate holder, a gas or gas mixture, a gas pressure, a voltage in pulse and a magnetic field so that the film to be deposited will contain the at least two chemical elements in multiple controlled oxidation states.

In a second aspect, the invention provides an apparatus for magnetically enhanced sputtering which comprises a process chamber. The process chamber contains a sputtering target, a substrate holder, a substrate to be coated, a gas inlet inside the process chamber and a power supply configured to apply a voltage in pulse between the sputtering target and the substrate to be coated and to generate a magnetic field. The apparatus is further characterized in that the sputtering target includes at least two different chemical elements.

In a fourteenth preferred embodiment, the sputtering target is further characterized in that the at least two different chemical elements are selected from the group consisting of transition metals, poor metals, metalloids or polyatomic nonmetals.

In a fifteenth preferred embodiment, the at least two different chemical elements are copper (Cu) and titanium oxide (TiO₂).

In a sixteenth preferred embodiment, the at least two different chemical elements are present in a ratio of 40 at. % for copper (Cu) and 60 at. % for titanium oxide (TiO₂).

In a seventeenth preferred embodiment, the process chamber is further characterized in that the substrate holder is mounted with mounting means in the process chamber so that a distance between the sputtering target and the substrate to be coated can be controlled.

In an eighteenth preferred embodiment, the distance between the sputtering target and the substrate to be coated is set at 10.5 cm.

In a nineteenth preferred embodiment, the gas is a mixture of an inert gas and a reactive gas.

In a twentieth preferred embodiment, the gas is a mixture of Argon and Oxygen.

In a twenty-first preferred embodiment, the mixture of Argon and Oxygen is in a ratio of FluxO₂/FluxAr=0.05.

In a twenty-second preferred embodiment, a voltage is applied in pulse between the sputtering target and the substrate to be coated so that the pulse has a power per pulse in a range of 1000 W to 2000 W and has a duration in a range of 50 μs to 200 μs.

In a twenty-third preferred embodiment, the apparatus is further characterized in that the power per pulse is 1750 W and the pulse has duration of 100 μs.

In a third aspect, the invention provides an active film as prepared with the inventive process.

In a twenty-fourth preferred embodiment, in the active film at least one of the at least two chemical element is in several oxidation states.

In a twenty-sixth preferred embodiment, the active film is a bioactive surface.

### BRIEF DESCRIPTION OF THE FIGURES

The invention will now be explained through the description of preferred embodiments while referring to figures, as listed herein below:

FIG. 1a illustrates the fastest bacterial inactivation leading to complete inactivation;

FIG. 1b illustrates the bacterial inactivation kinetics by TiO₂ sputtered samples;

FIG. 1c illustrates the E. coli inactivation within 60 min for high power impulse magnetron sputtering Cu-sputtered samples within 15, 30, and 60 s;

FIG. 1d illustrates the results for the diffuse reflectance spectrometry for the TiO₂/Cu samples used to evaluate the bacterial inactivation (FIG. 1a);

FIG. 1e illustrates the E. coli survival on TiO₂/Cu HIPIMS-sputtered sample for 150 s up to the 8th repetitive cycle under solar simulated light.

FIG. 1f illustrates, the release of Cu-ions inactivating E. coli as a function of the catalyst recycling;

FIG. 2a illustrates the atomic percentage concentration of Cu, Ti, O₂ and C of TiO₂/Cu samples sputtered for 150 s as a function of depth penetration of the Ar-ions;

FIG. 2b illustrates the 3-D view of the Cu2p3/2 doublet and the Cu shake-up satellites at 933.4 eV and at 933.1 eV for the TiO₂/Cu 150 s high power impulse magnetron sputtering sample;

FIG. 2c illustrates the Ti2p3/2 doublet peaks with binding energies (BE) at 458.5 and 464.1 eV, increasing steadily as we go deeper into the TiO₂/Cu film up to ˜125 layers;

FIG. 2d illustrates the XPS envelope for the Ti2p signals;

FIG. 2e illustrates the XPS envelope for the Ti2p signals;

FIG. 2f illustrates the CuO initial decreases while concomitantly the Cu₂O grows in line with the redox catalysis taking place in TiO₂/Cu shifting the CuO peak;

FIG. 3 illustrates the interfacial charge transfer between TiO₂ and Cu;

FIG. 4a illustrates the loss of viability time vs thickness for DCP and high power impulse magnetron sputtering TiO₂/Cu sputtered films;

FIG. 4b illustrates a scheme for the DC, DCP and HiPIMS sputtering proceedings showing a difference in the ionisation of the sputtered species in the process chamber;

FIG. 5 illustrates a scheme of a process chamber,

table 1 represents the content of TiO₂ and CuO with increased sputtering time;

table 2 represents a constant atomic percentage concentration implying that a rapid catalytic decomposition of the bacterial residues on the sample surface;

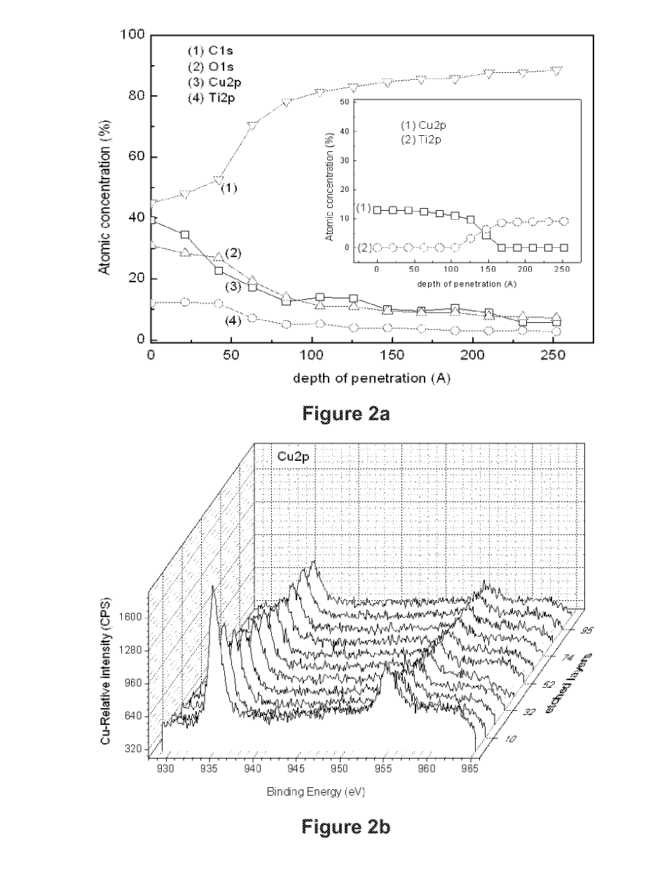

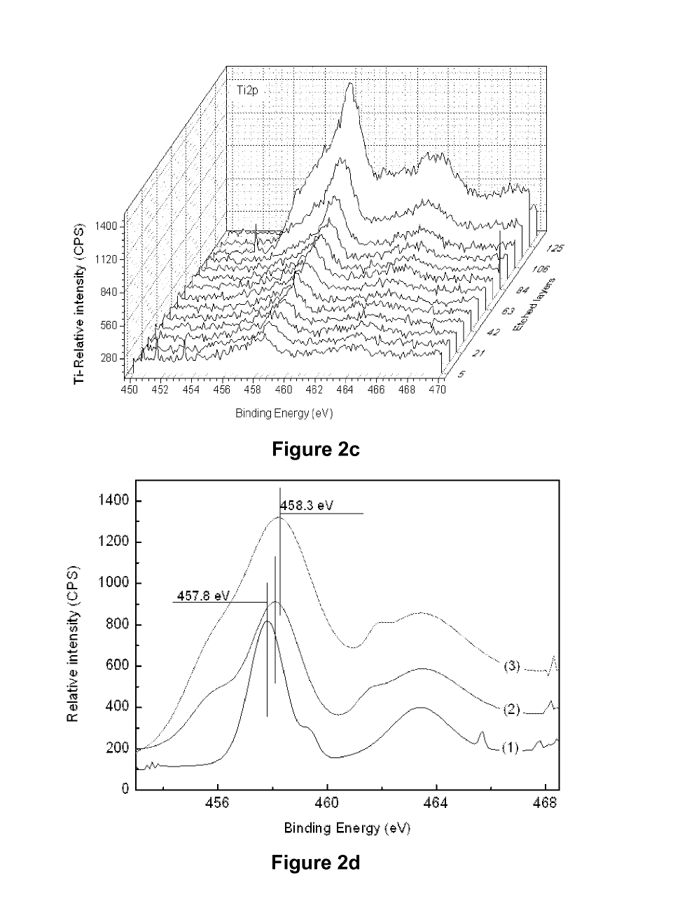

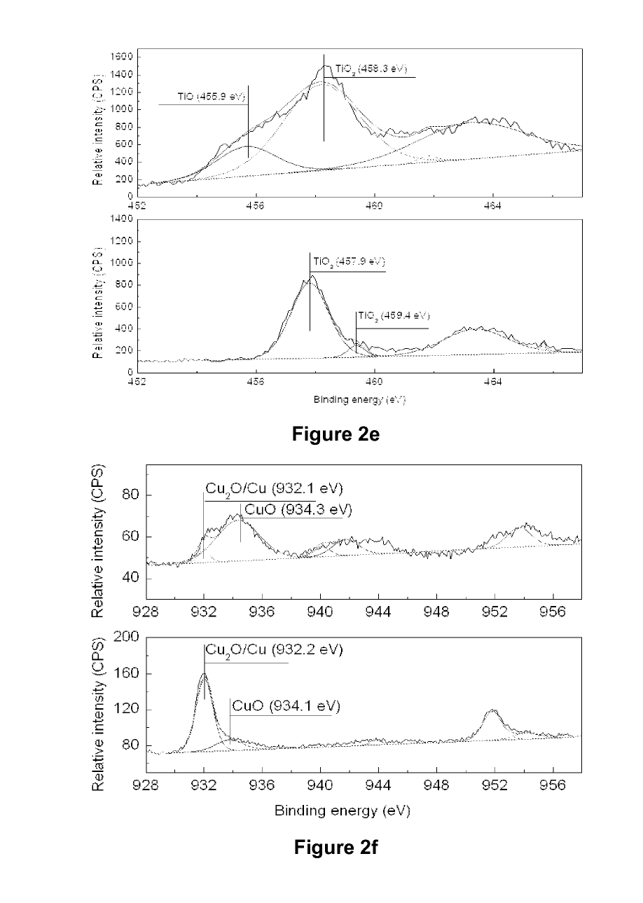

table 3 represents a significant growth of the Cu₂O peak as detected in FIG. 2g.

### DESCRIPTION OF PREFERRED EMBODIMENTS

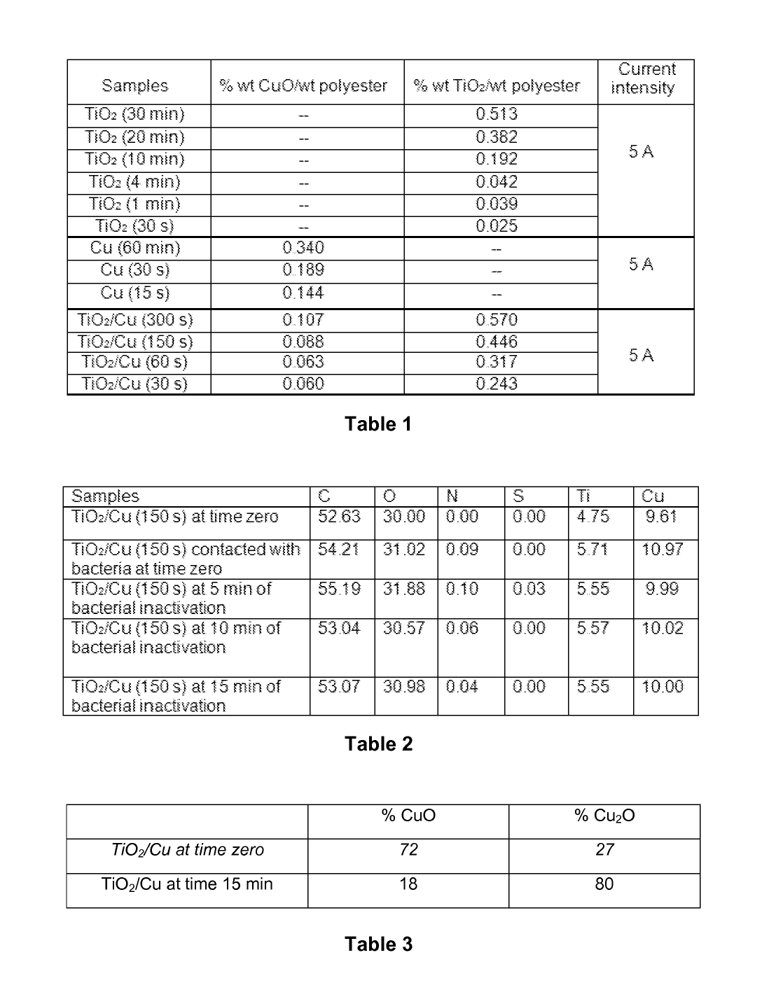

In one embodiment, the present invention relates to an optimised high power impulse magnetron sputtering on 3D substrates A leading to ultrathin uniform films showing an accelerated bacterial inactivation. Due to the induced high energy Cu-ions (M+) produced in the process chamber E, illustrated in FIG. 5, the high power impulse magnetron sputtering plasma C density and the increased effect of the applied bias voltage on the Cu-ions (M+) sputtered by high power impulse magnetron sputtering compared to DC/DCP sputtering. Thin and adhesive Cu and TiO₂/Cu films sputtered by high power impulse magnetron sputtering on polyester samples present the potential to be practical candidates to avoid biofilm formation and disinfect hospital rooms not involving a high level of bacterial concentration [1-5].

The process according to the present invention utilizes a process gas; ideally this process gas is a mixture of an inert gas and a reactive gas. Inert gases are ideally noble gases or nitrogen. Reactive gases such as oxygen, ozone, halogen gases, oxidised nitrogen compounds, sulphur dioxide, ammonia, phosphine, volatile organic compounds among others can be used in relation to the nature of the requested composition of the active film.

The high-power impulse magnetron sputtering (HIPIMS) discharge is a type of high-current plasma glow, which is typically characterized by a high voltage of 400-2000 V and a high-current density of 0.1-10 A/cm². HIPIMS discharges are homogeneously distributed over the cathode area. The intermediate stage of the gas breakdown process occurs at a few hundred volts and high-current density of several A/cm² that could only be sustained over a limited period. The gas transits from low ionization directly to the quasi-stationary state and after a time period transits to the higher current density arc stage. The Ar and metal atoms were ionized and that double-charged metal ions were present as detected by plasma sampling mass spectroscopy B to show that the metal ionization reaches up to 70%. HIPIMS operates at significantly lower pressure of <10 m Torr, which is desired to allow efficient discharge around ˜200 Hz so that the average power of the discharge remains within standard cathode cooling. A plasma density >10¹³ cm³ rich in metal ions is established near the substrates A. The HIPIMS discharge is sustained by secondary electron emission by similar mechanisms as a conventional magnetron discharge. It is distributed homogeneously over the surface of the cathode.

HIPIMS is a stable discharge and has been demonstrated to work with a variety of elements such as transition metals, poor metals, metalloids or polyatomic nonmetals (B, C, Al, Si, Sc, Ti, V, Cr, Cu, Zn, Y, Zr, Nb, Mo, Ag, Ta, W and Au among others). At higher powers, the plasma density at the position of the substrate A increases faster than at low powers possibly due to the escape of plasma C from the target confinement, extension of the ionization.

Hereafter the process is explained in regard of Copper, but it is to be understood that the same would apply for chemical elements with multiple potential oxidation states in the adapted process condition. The formation in the process chamber E of Cu(0), Cu(+1), Cu(+2), Cu(+3) or Cu(+4) can be understood in terms of:
a) The partial oxidation of Cu in the process chamber E in the presence of an oxygen source. This source of oxygen is the residual H₂O vapor in the process chamber E at the residual pressure Pr=10⁻⁴ Pa. This pressure is representative of about 10¹⁵ molecules/cm². Therefore, there are sufficient oxygen radicals available in the process chamber E to induce a variable oxidation of the Cu.
b) The atoms sputtered during Ar bombardment of the target enter in collision with other atoms present during the process (gas atmosphere). The probability of collision between the particles is governed by the plasma density, the Ar flux and the sputtering yield of the target. During its course to the substrate A, the sputtered atom by HIPIMS has a reduced mean free path compared to DC and DCP (Mean free path is the average distance that an atom can move in one direction, without colliding at another atom).
c) The Cu films readily oxidize after sputtering when exposed to ambient air. Therefore a variable oxidation of Cu could be observed by XPS depending on the experimental conditions used during the HIPIMS deposition and after the deposition.

By controlling and adapting the different parameters during the sputtering process, the population of the chemical element in different oxidation state, i.e. Cu, can be controlled.

In one embodiment of the invention, high power impulse magnetron sputtering deposition of Ti and Cu is carried out in Vacuum system at 5.8×10⁻³ mbar. The Cuas well as the TiO₂/Cu sputtering targets D are 50 mm in diameter, 99.99% pure. The TiO₂/Cu target is 2 inches in diameter and has a composition of 60/40 atomic % in TiO₂ and Cu respectively. The high power impulse magnetron sputtering is operated at 500 Hz with pulses of 100 microseconds separated by 1.9 ms, this leading to a deposition rate for TiO₂/Cu of 15.3 nm/min. The average power is 87.5 W (5 A×350 V) and the power per pulse of 100 microseconds is 1750 W. The 5 A current is the current at one pulse, the voltage at one pulse is 350V and the pulses had a rectangular shape since the pulse duration is 100 microseconds with an off period of 1900 microseconds and up.

In another embodiment, the DCP of 622 V and 0.3 A is applied during the 3 pulses of 10 microseconds each within a 50 microsecond period. This gives 187 W per period or 62.3 W/pulse and an average power of 312 W/period.

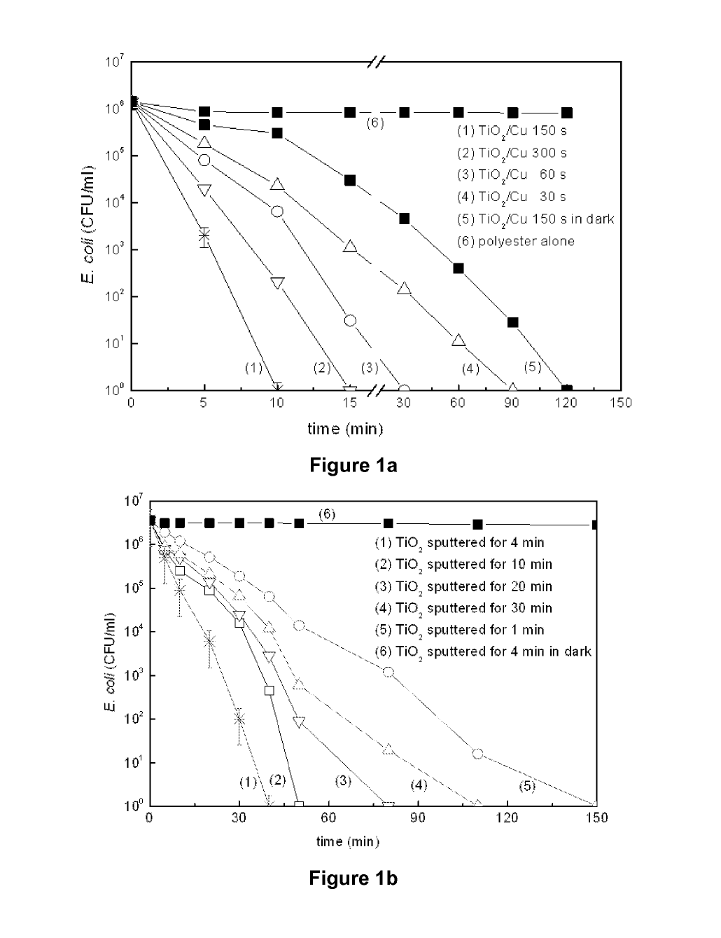

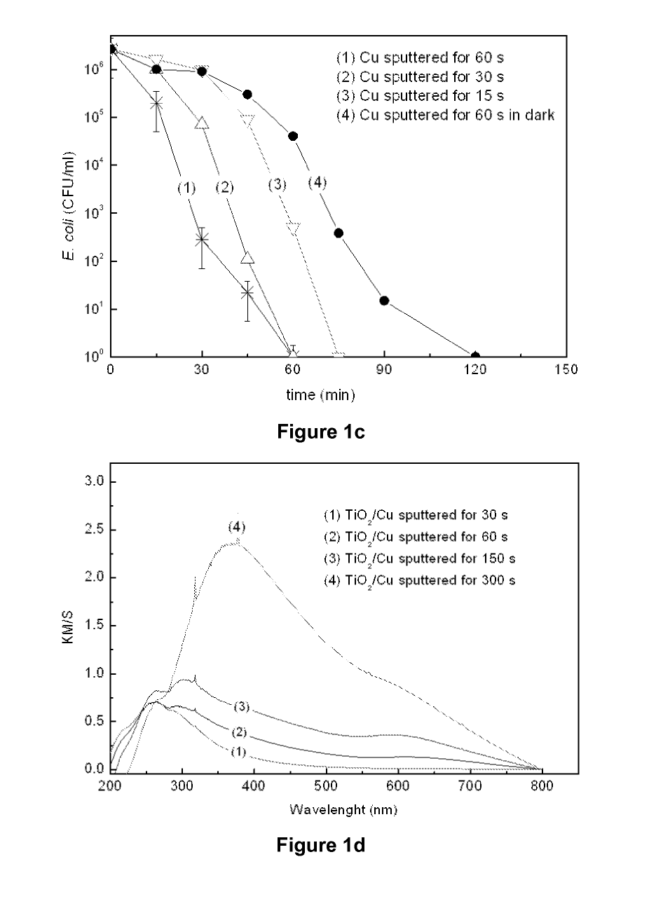

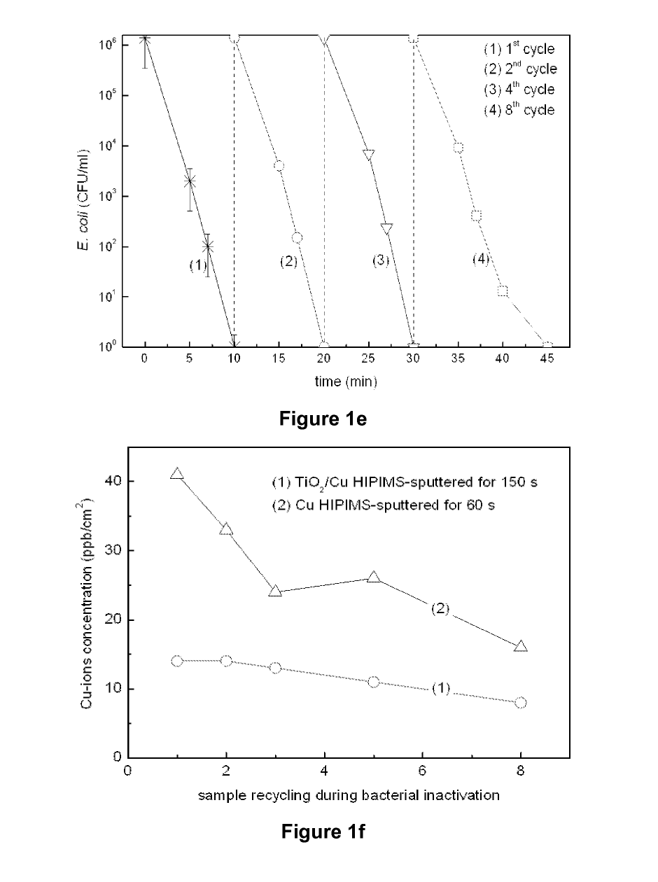

The calibration of the Cu-nanoparticulate film thickness by high power impulse magnetron sputtering on the Si-wafers is shown in FIG. 1a. The film thickness can be determined with a profilometer. The detection of the oxidative species (mainly OH-radicals) in the TiO₂/Cu sputtered samples can be carried out according to Ishibashi et al., [19].

The thickness calibration for Cu, TiO₂ and TiO₂/Cu 60%/40% (from mixed target D) HIPIMS sputtered on Si-wafers at 5 A was investigated. The fastest bacterial inactivation leading to complete inactivation was observed when the polyester sputtered for 150 s with the TiO₂/Cu sputtering target D (FIG. 1a) depositing a composite film 38 nm thick. This is equivalent to ˜190 layers 0.2 thick nm with 10¹⁵ atoms/cm² and deposited at a rate of 15.3 nm/min or 7.6×10¹⁶ atoms/cm²/min. X-ray fluorescence in Table 1 shows the content of TiO₂ and CuO with increased sputtering time. When using the TiO₂/CuO 60%/40% sputtering target D a ratio of TiO₂/CuO of 4-5 times was observed for the different sputtering times.

The bacterial loss of viability in FIG. 1a, trace 6 shows that no bacterial loss of viability occurs on polyester alone under light irradiation. Runs the dark for samples sputtered for 150 s induced a slow loss of bacterial viability within 120 min, showing that the bacterial CFU reduction involves Cu-layers. Under actinic light radiation, traces 3 and 4 indicate that sputtering times of 30 s and 60 s induce faster bacterial loss of viability kinetics. A sputtering time of 150 s induced the shortest inactivation time (trace 1). Sputtering for 300 s induce bacterial inactivation taking longer times compared to samples sputtered for 150 s. Therefore, the amount of Cu⁰ is not the main species leading to bacterial inactivation. A sputtering time of 150 s is seen to leads to the most favourable structure-reactivity for the Cu-polyester leading to the shortest E. coli inactivation. This sample presents the highest amount of Cu-sites held in exposed positions interacting on the surface or close to the polyester surface with E. coli leading to bacterial loss of viability [17a]. The surface bactericide action seems to be due to a synergic effect introduced by the TiO₂/Cu layers since longer times were observed when sputtering TiO₂ as shown next in FIG. 1b.

FIG. 1b shows the bacterial inactivation kinetics by the high power impulse magnetron sputtering TiO₂ sputtered samples. As shown in FIG. 1b no bacterial inactivation takes place in the dark but the bacterial inactivation becomes faster for high power impulse magnetron sputtering times between 1 min (trace 5) and 4 min (trace 2). Longer deposition times between 10 and 30 min did not accelerate the loss of viability due to the fact that an increased TiO₂ thickness >12 nm sputtered within 4 min leads to:
a) bulk inward diffusion of the charge carriers generated on TiO₂ under light leading to highly oxidative radicals [20-21], and
b) longer sputtering times facilitate the TiO₂ inter-particle growth decreasing the TiO₂ contact surface with bacteria [14-15].

The TiO₂ bactericide inactivation mechanism has been reported and will not be discussed further in the present description [6-7,20]. FIG. 1c shows the E. coli inactivation within 60 min for high power impulse magnetron sputtering Cusputtered samples within 15, 30, and 60 s. This inactivation time is longer than the time reported in FIG. 1a suggesting a synergic effect between TiO₂ and Cu leading to a faster bacterial loss of viability.

FIG. 1d presents the results for the diffuse reflectance spectroscopy (DRS) for the TiO₂/Cu samples used to evaluate the bacterial inactivation (FIG. 1a). The absorption in Kubelka-Munk units shows agreement with the data reported for TiO₂ and Cu Table 1, showing that TiO₂ is the main surface element. The Cu/Cu₂O/CuO absorption increases with longer Cu-sputtering times up to 300 s [22]. The weak absorption from 400 and 500 is due to the interfacial charge transfer (IFTC) from the TiO₂ to CuO. The optical absorption between 500 and 600 nm is due to the interband transition of Cu₂O. The absorption between 600 to 800 nm has been attributed to the exciton band and the Cu(II) d-d transition.

The rough UV-Vis reflectance data cannot be used directly to assess the absorption coefficient of the sputtered polyester because of the large scattering contribution to the reflectance spectra. Normally, a weak dependence is assumed for the scattering coefficient S on the wavelength. The KM/S values for the samples in FIG. 1d are proportional to the TiO₂/Cu absorption coefficient up to sputtering times of 150 s and these values are in agreement with the trend observed during the bacterial inactivation kinetics reported in FIG. 1a.

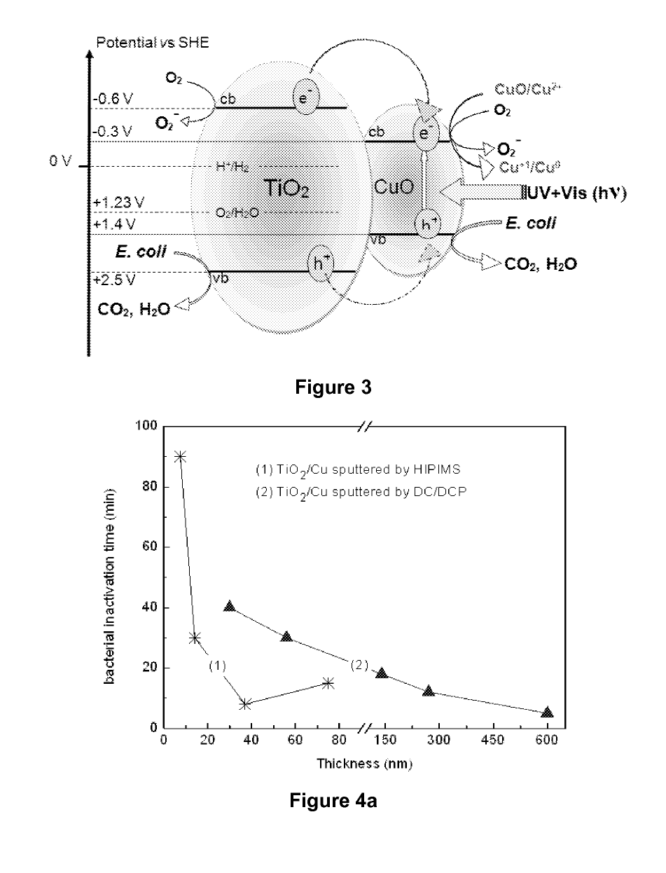

The loss of bacterial viability due to the TiO₂/Cu sample irradiated by three different light doses in the solar simulator was investigated. The loss of bacterial viability with time is shown to be a function of the intensity of the applied visible light. The mechanism will be discussed below in the section describing the results presented in FIG. 3.

FIG. 1e shows the recycling of the TiO₂/Cu (150 s) sample up to the 8th cycle. No loss in activity was observed in the sample during the sample recycling. The sample was thoroughly washed after each recycling leading to the reuse of the sample since complete bacterial loss of viability was attained after each cycle. The chemical state and environment of the CuO/Cu-ions seem not to change after the bacterial loss of viability showing the stable nature of the TiO₂/Cu on the polyester fabric.

FIG. 1f shows the release of Cu-ions inactivating E. coli as a function of catalyst recycling. FIG. 1f shows the repetitive release of Cu-ions up to the 8th recycling as measured by ICP-MS. The release of Cu− from the TiO₂/Cu samples shown in FIG. 1f was ˜8 ppb/cm². This value is lower compared to the Cu-release from the Cu-sputtered samples reaching up to ˜18 ppb Cu/cm² at the end of the 8th cycle. In both cases the small amounts of Cu are considered not to be cytotoxic to mammalian cells and proceed through an oligodynamic effect [6,17]. The Cu and TiO₂/Cu induced bacterial inactivation is carried out in a way that it is not toxic to human health.

The particle size of the film nanoparticulate and the hydrophobic-hydrophilic balance determine to great extent the surface photocatalytic properties. Samples sputtered for 30 s show Cu-nanoparticles between 8-15 nm. The TiO₂ samples sputtered for 150 s present sizes between 8-12 nm, and the TiO₂/Cu samples sputtered for 150 s presented particles 5-10 nm. The TiO₂ binds, disperse and stabilize the Cu-clusters on the polyester surfaces. The nanoparticles small size accounts for the favorable bacterial inactivation kinetics due to the large surface area per unit mass [14-15, 20, 23]. The distribution of TiO₂ and Cu-nanoparticles on the polyester was found to be uniform not presenting any cracks. The uniformity of the film is beneficial for the bacterial adhesion which is the primary step leading to the bacterial loss of viability to proceed favorably [1-2, 8]. The electronic transfer between the TiO₂/Cu sample and the E. coli depends on the length of the charge diffusion in the composite film. This in turn is a function of the TiO₂ and Cu particle size and shape [20-21].

The interfacial distances between TiO₂ and Cu/CuO on the polyester surface range below 5 nm. This allows the interfacial charge transfer (IFCT) to proceed with a high quanta efficiency [20, 23]. Quantum size effects have been shown to occur in particles with sizes 10 nm having about 10⁴ atoms as presented by the TiO₂ particles with sizes ˜10 nm [23-24]. But in the CuO nanoparticles the charge recombination increases within shorter times due to the decrease in the available space for charge separation. Also, the decrease of the space charge layer decreases further the potential depth.

The Cu-nanoparticles are observed to be immiscible with Ti. Cu²⁺ and does not substitute Ti⁴⁺ in the TiO₂ lattice because of the significant difference in the radii of Ti⁴⁺ (0.53 Angstrom) and Cu²⁺ (1.28 Angstrom). Due to its size, the CuO/Cu nanoparticles with particle size >8 nm are not able to penetrate into the bacteria core through the cell wall pores with diameters of 1-1.3 nm [25]. Only Cu-ions diffuse through bacterial pores leading to DNA damage and finally to the total loss of bacterial viability.

The surface atomic percentage composition of C, O, N, S, Ti and Cu is shown in Table 2 as a function of bacterial inactivation time when using HIPIMS sputtered samples up to 15 min. Table 2 shows a constant atomic percentage concentration implying that a rapid catalytic decomposition of the bacterial residues on the sample surface. Within 15 min the bacterial residues are destroyed enabling the catalyst recycling as shown in FIG. 1g.

FIG. 2a presents the atomic percentage concentration of Cu, Ti, O₂ and C of TiO₂/Cu samples sputtered for 150 s as a function of depth penetration of the Ar-ions. It is readily seen that Cu, Ti and O decrease up to 240 Angstroms due to the Ar-bombardment. The etching depth induced by the Ar-ions was referenced by the known etching value for Ta of 15 atomic layers per minute equivalent to ˜30 Angstroms/min. The penetration of the Cu inside the sample protects the Cu-clusters inside the 130 microns thick polyester network during the E. coli inactivation process. The increase in the C-content in FIG. 2a is due to the etching removing the TiO₂/Cu layers making available the C-content of the polyester. The insert in FIG. 2a shows the significantly lower percentage of Cu and Ti for TiO₂/Cu sputtered by DC/DCP [17]. The concentration of Ti followed a different pattern compared to the one observed when sputtering by high power impulse magnetron sputtering and increases beyond 100 Angstroms because Ti deposition was hindered by the Cu-layers. FIG. 2b presents the 3-D view of the Cu 2p3/2 doublet and the Cu shake-up satellites at 933.4 eV and at 933.1 eV [18a] for the TiO₂/Cu 150 s high power impulse magnetron sputtering sample. The Cuenrichment within the 10 upper layers is seen to decrease with sample depth and remain stable up to ˜100 layers. FIG. 2c shows the Ti 2p3/2 doublet peaks with binding energies (BE) at 458.5 and 464.1 eV, increasing steadily as we go deeper into the TiO₂/Cu film up to ˜125 layers.

FIG. 2d presents the XPS envelope for the Ti2p signals at zero, 5 min and 10 min shown in the traces (1) through (3). It is readily seen that redox Ti³⁺/Ti⁴⁺ processes take place during bacterial inactivation shifting the peak from 457.8 to 458.3 eV. This is >0.2 eV accepted as a true change in the oxidation state of a specific species [15,18a]. FIG. 3e present the deconvolution of the peaks for the Ti2p doublet before and after the bacterial inactivation process. Evidence is presented for the reduction from Ti(iV) to Ti(III) in FIG. 3 by the shift of the deconvoluted peak from 457.9 eV at time zero to 458.3 eV after 10 min, the end of the bacterial inactivation.

Evidence is presented in FIGS. 3f-3g by XPS for Cu-redox chemistry during the bacterial inactivation in addition to the redox chemistry described above in FIGS. 3d and 3e for Ti³⁺/Ti⁴⁺ states. The experimental envelope for the XPS peaks at time zero for CuO was seen at 934.3 eV and for Cu₂O at 932.1 eV. The Cu₂O peak in TiO₂/Cu grows during the bacterial inactivation after 5 minutes and after 15 min when the bacterial when the inactivation is complete. In agreement with Table 3, a significant growth of the Cu₂O peak is detected in FIG. 2f due to two reasons:
a) the CuO initial decreases from 72% to 18% while concomitantly the Cu₂O grows from 27% to 80% in line with the redox catalysis taking place in TiO₂/Cu shifting the CuO peak in FIG. 2f to CuO 934.1 eV and
b) the bacteria covering initially the TiO₂/Cu catalyst has been removed during the inactivation process.

It can be suggested that the interactions between Cu+/Cu²⁺ and Ti³⁺/Ti⁴⁺ in the TiO₂/Cu samples play an active role accelerating the bacterial inactivation. The Ti³⁺/Ti⁴⁺ surface electron sites enhance the O₂ chemisorption at the surface more markedly in the TiO₂/Cu samples. This leads to a fast bacterial inactivation by TiO₂/Cu compared to Cu in FIG. 1d. The hole transition from TiO₂vb to the Cu mid band-gap states is in a second stage followed by indirect electronic transitions from the mid-gap states reaching the TiO₂cb.

FIG. 3 shows the interfacial charge transfer between TiO₂ and Cu in the TiO₂/Cu photocatalyst TiO₂/Cu under simulated solar irradiation. In the TiO₂ semiconductor the solar irradiation induces both the e⁻ transfer and h⁺ transfer from TiO₂ to CuO since the potential energy levels of the TiO₂cb and TiO₂vb lie above the CuOcb and CuOvb levels. The partial recombination of e−/h+ in the TiO₂ is hindered by the transfer of charges to the CuO facilitating the reactions occurring at the TiO₂cb and CuOcb as shown in FIG. 3. Under simulated solar light as shown in FIG. 3, the CuO can be reduced to Cu₂O and the Cu₂O can reduce O₂ via a multi-electron process and re-oxidize to CuO. The charges generated by light in the TiO₂/Cu lead to the rapid loss of E. coli viability ≦10 min (FIG. 1a), along O₂ and CuO reduction at the CuOcb as suggested in FIG. 3.

The interfacial charge transfer (IFCT) in the TiO₂/Cu sample seems to proceed with high quantum efficiency under light irradiation since the bacterial inactivation proceeds within short times 10 min (FIG. 1a). But the magnitude of the increase in the IFCT absorption of the TiO₂/Cu shown by the DRS spectra in FIG. 1e is relatively small.

The conduction band of CuO at −0.30 V vs SCE (pH 7) is at a more negative potential than the potential required for the one electron oxygen reduction O₂+H⁺+e⁻ →HO₂°−0.22 V [25-26]. Furthermore, the Cu²⁺ can react with e− (or O₂)→Cu++(or O₂). The Cu+ can reduce O₂ consuming electrons or be reoxidized to Cu²⁺ by the photo-generated TiO₂ holes [27]. The TiO₂vb holes react with the surface —OH of the TiO₂ releasing OH-radicals to inactivate bacteria [28].

The fluorescence intensity of the TiO₂/Cu HIPIMS-sputtered samples irradiated up to 15 min in the solar simulator was investigated. The OH-radicals originate from the reaction between the OH-radical and terephthalic acid leading to formation of a fluorescent hydroxy-product [19]. The TiO₂ vb holes in FIG. 3 have the potential to degrade polyester during the bacterial inactivation cycles. But the stable repetitive E. coli loss of viability reported in FIG. 1e shows that bacterial inactivation did not lead to the degradation of polyester up to the 8th recycling.

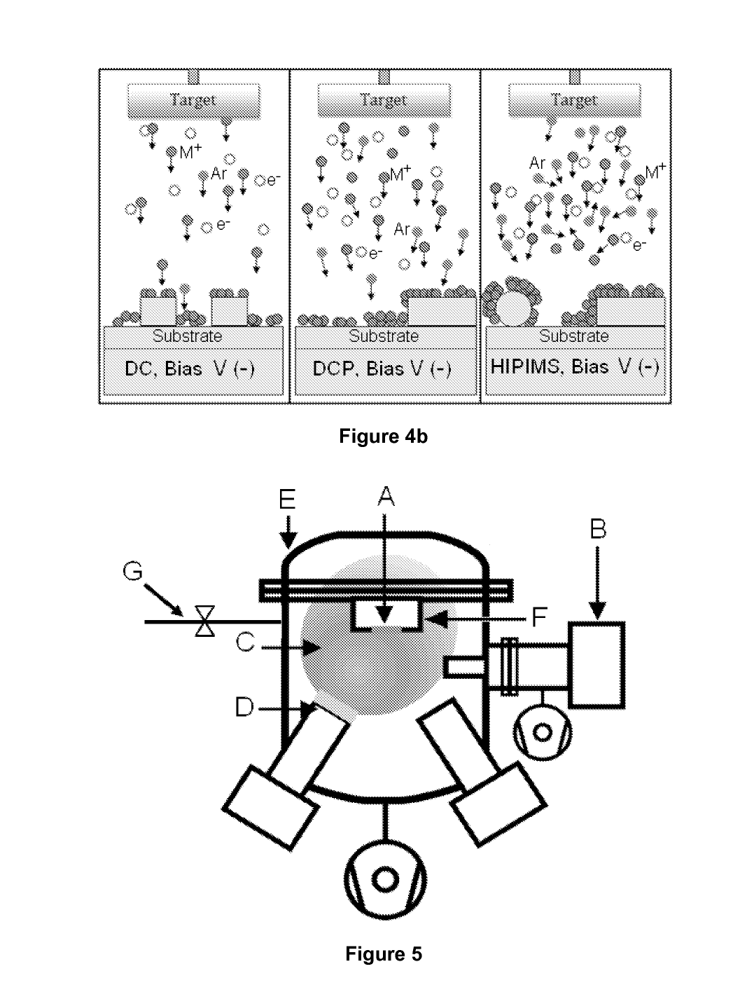

FIG. 4a presents the loss of viability time vs thickness for DCP and high power impulse magnetron sputtering TiO₂/Cu sputtered films. FIG. 4a shows the much thinner TiO₂/Cu layer thickness necessary for complete bacterial inactivation on HIPIMS sputtered samples compared to samples sputtered by DC/DCP. FIG. 4a shows that the high power impulse magnetron sputtering film with a thickness of 38 nm inactivated bacteria within ˜10 min compared to a sputtered DC/DCP film 600 nm thick inducing inactivation bacterial inactivation within the same period of time.

In FIG. 4b, left hand side presents a scheme for the DC sputtering proceeding with an ionization of the Cu-ions of 1% [29]. The DCP sputtering is schematically presented in FIG. 4b (middle section) and proceeds with ionization of Cu-ions well above the values attained by DC [30]. FIG. 4b, right hand side involves high power impulse magnetron sputtering leading to a Cu-ionization of ca. 70% and an electronic density of ˜10¹⁸⁻¹⁹ e−/m³ [31]. The high power impulse magnetron sputtering power per pulse was 1750 W/100 microseconds. This value is significantly higher than the power per pulse applied by DCP of 62.3 W/10 microseconds. The high power impulse magnetron sputtering higher energy increased the ionization percentage Cu^{o}→Cu⁺/Cu²⁺.

This increased arrival energy of the Cu-ions on the substrate A allows the alignment of the Cu-ions on the polyester irregular (rough) surface enabling a uniform coverage of the 3-D polyester. The polyester 3-D presents roughness could not be quantified by atomic force microscopy (AFM) since it is beyond the AFM experimental range of 10 microns.

The present description presents the first evidence for the surface functionalization of polyester by HIPIMS sputtered thin layers of TiO₂/Cu able to inactivate bacteria in the minute range. The TiO₂/Cu thin films were uniform, presented adhesive properties and led to repetitive loss of bacteria viability. A faster inactivation kinetics was observed by the TiO₂/Cu films compared to Cu or TiO₂ sputtered separately. A polyester sample high power impulse magnetron sputtering sputtered for 10 min at 5 A led to a complete inactivation 10 min under solar simulated light irradiation.

A considerable saving in metal and deposition time (energy) was found with high power impulse magnetron sputtering compared to conventional DC/DCPsputtering on 3-D surfaces. The increasing demand for Cu is decreasing rapidly the known world reserves. This is important since Cu is a strategically important metal. High power impulse magnetron sputtering films of TiO₂/Cu and Cu on polyester have been shown in this study to preclude biofilm formation in the dark and more significantly under light irradiation.

### REFERENCES

[1] K. Taylor. R. Roberts, J. Roberts, J. The challenge of hospital acquired infections (HAI), Nat. Audit Office, 2002.
[2] S. Dance, S. The role of environmental cleaning in the control of hospital acquired infections J. Hosp. Infect. 73 (2007) 378-389.
[3] G. Borkow, and J. Gabbay, Puttting copper into action. Copper impregnated products with potental biocidal activities. J. FASEB, 188 (2008) 1728-1730.
[4] A. Torres, C. Ruales, C. Pulgarin, A. Aimable, P. Bowen, J. Kiwi, Enhanced Inactivation of E. coli by RF-plasma Pretreated Cotton/CuO (65 m2/g) under Visible Light, Appl. Mater, Interf, 2 (2010) 2547-2552.
[5] L. Zhang R. Dillert, and D. Bahnemann, Photoinduced hydrophylicity and selfcleaning: models and reality, En & Environ. Sci, 5 (2012) 7491-7507.
[6a] K. Sunada, Watanabe, K. Hashimoto, Bactericidal Activity of Copper-Deposited TiO2 Film under UV Light Illumination, Environ & Environ. Sci Technol. 37 (2003) 4785-4789.
[6b] O. Akhavan, R. Azimiriad, S. Safa, E. Hasani, CuO/Cu(OH)2 hierarchical nanostructures as bactericidal photocatalysts, J. Chem. Mater. 21 (2011) 9634-9640.
[6c] O. Akhavan, E. Ghaderi Copper-oxide nanoflakes as highly sensitive and fast response self-sterilizing biosensors, J. Chem. Mater. 21 (2011)12935-12940.
[6d] O. Akhavan, E. Ghaderi, Cu and CuO nanoparticles immobilized by silica thin films as antibacterial materials and catalysts, Surf. & Coatings Technol. 205 (2010) 219-223.
[7] H. Irie, S. Miura, K. Kamiya K. Hashimoto, Efficient visible light-sensitive photocatalysis: Grafting Cu(II) ions onto TiO₂ and WO₃ photocatalyssts, Chem. Phys. Letts, 457 (2008) 202-205.
[8] K. Page, M. Wilson, P. I. Parkin, Antimicrobial surfaces and their potential in reducing the role of the inanimate environment in the incidence of hospitalacquired infections, J. Mater. Chem. 1 (2009) 3819-3831.
[9] H. A. Foster, P. Sheel, W. D. Sheel, P. Evans, S. Varghese, N. Rutschke, M. H. Yates, Antimicrobial activity of titatnia/silver and titania/copper films prepared by CVD, J. Photochem. Photobiol. A. 216 (2010) 283-289.
[10] M. S. P. Dunlop, P. C. Sheeran, A. J. M. Byrne, S. A. McMahon, M. A. Boyle, G. K. McGuigan, Inactivation of clinically relevant pathogens by photocatalytic coatings, J. Photochem. Photobiol. A. 216 (2010) 303-3010.
[11] M. H. Yates, A. L. Brook, B. I. Ditta, P. Evans, H. A. Foster, D. W. Sheel, A. Steele, Photo-induced self-cleaning and biocidal behviour of titania and copper oxide multilayers, J. Photochem. Photobiol. A. 197 (2008) 197-2008.
[12] J. Lin, J. Moore, W. Sproul, B. Mishra, Z. Wu, L. Wang, The structure and properties of chromium nitride coatings deposited using dc, pulsed dc and modulated pulse power magnetron sputtering, Surf & Coat. Technol. 204 (2010) 2230-2239.
[13] Sarakinos, K.; Alami, J.; Konstantinidis, D. High power pulsed magnetron sputtering: A review on scientific and engineering state of the art, Surf & Coat. Technol. 204 (2010) 1661-1684.
[14] P. Osorio, R. Sanjines, C. Ruales, C. Castro, C. Pulgarin, J-A Rengifo, J-C Lavanchy, J. Kiwi, Antimicrobial Cu-functionalized surfaces prepared by bipolar asymmetric DC-pulsed magnetron sputtering (PMS), J. Photochem. Photobiol. A., 220 (2011) 70-76.
[15] E. Kusiak-Nejman, A. Morawski, A. Ehiasarian, O. Baghriche, C. Pulgarin, E. Mielczarski, J. Mielczarski, A. Kulik, J. Kiwi, E Coli Inactivation by High Power Impulse Magnetron Sputtered (HIPIMS) Cu-Surfaces, J. Phys. Chem. C., 115 (2011) 21113-21119.
[16] 0. Baghriche, S. Rtimi, C. Pulgarin, T. Roussel, J. Kiwi, Effect of the spectral properties of TiO₂, Cu, TiO₂/Cu sputtered films on the bacterial inactivation under low intensity actinic light J. Photochem. Photobiol. A, 213 (2013) 50-59
[17] a) S. Rtimi, O. Baghriche, C. Pulgarin, R. Sanjines, J. Kiwi, Innovative TiO₂/Cu surfaces inactivating bacteria <5 min under low intensity visible/actinic light ACS Appl. Mater. & Interf. 4 (2012) 5234-5240,
b) L. Rio, E. Kusiak, J. Kiwi, C. Pulgarin, A. Trampuz, A. Bizzini, Comparative methods to evaluate the bactericidal activity of copper-sputtered surfaces against methicillin-resistant Staphylococcus aureus, J. Appl. Microb. 78 (2012) 8176-8182.
[18] a) D. Wagner, M. Riggs, E. Davis, G. Mullenberg (Eds), Handbook of X-ray Photoelectron spectroscopy, Perkin-Elmer Corporation Physical Electronics Division, Minnesota, 1979.
b) D. Shirley, Corrections of electrostatic charged species in XPS-spectroscopy, Phys. Rev. B5 (1972) 4709-4716.
[19] K. Ishibashi, A. Fujishima T. Watanabe, K. Hashimoto, Detection of active oxidative species in TiO2 photocatalysis using the fluorescence technique, Electrochem. Comm. 2 (2000) 207-2010.
[20] W. Tung, W. Daoud, Selfcleaning fibers via nanotechnology: a virtual reality J. Mat. Chem. 21 (2011) 7858-7869.
[21] I. Mathews, Epitaxial Growth Part b, IBM, Academic Press, New York, 1975, p. 382-436.
[22] K. Hardee, A. Bard, Electrodes, X. Photochemical Behavior of Several Polycrystalline Metal Oxides Electrodes in Aqueous Solutions, J. Electrochem. Soc. 124 (1977) 215-224.
[23] V. Nadtochenko, V. Denisov, O. Savinov, J. Kiwi. Laser kinetic spectroscopy in the interfacial charge transfer between membranes cellwall, J. Photochem. Photobiol. A, 181 (2006) 401-407.
[24] J. Kiwi, C. Morrison, Dynamics of Charge Transfer on Li-doped Anatase based Catalyst powders with Enhanced Water Photo-cleavage under UV-irradiation, J. Phys. Chem. 88 (1984) 6146-6172.
[25] J. H. Nikaido, Prevention of Drug Access to Bacterial Targets. Permeability Barriers and Active Flux, Biol. Chem. 269 (1994) 3905-3909.
[26] A. Nozik, Photo-electrochemistry: Applications to Solar Energy Conversion, Annual Rev. Phys. Chem. 2 (1978)189-222.
[27] J. Bandara, I. Guasaquillo, P. Bowen L. Soare, W-F Jardim J. Kiwi, Photocatalytic Storing of O2 as H2O2 Mediated by High Surface Area CuO. Evidence for the Reductive-Oxidative Interfacial Mechanism of Reaction, Langmuir, 21 (2005) 8554-8559.
[28] D. Ward, A. Bard, Photocurrent enhancement via trapping of photo-generated electrons of titanium dioxide particles, J. Phys. Chem. 86 (2004) 3599-3604.
[29]. Petrov, A. Myers, J. E. Greene and J. R. Abelson, Mass and energy resolved detection of ions and neutral sputtered species incident at the substarte during reactive magnetron sputtering of Ti and mixed Ar+N2 mixtures, J. Vac. Sci. Technol. A 12(1994) 2846-2851.
[30] J. Alami, P. Persson, J. Gudmunsoon, J. Bohlmark, J. Helmersson J., Ionassisted physical vapor deposition for enhanced film properties on nonflat surfaces J. Vac. Technol. A, 23 (2005) 278-280.
[31] V. Kousznetsov, K. Macak, J. Schneider, U. Helmersson, I. Petrov, Surf. Coat. Technol. 12 (1999) 290-295.
[32] S. Rossnagel, & J. Hopwood, Magnetron sputter deposition with high levels of metal ionization J., Appl. Phys. Letts, 63 (1993) 32-34.

1-26. (canceled)

27. A process for depositing a film onto a complex three-dimensional (3D) substrate, comprising steps of:
inserting into a process chamber a sputtering target including at least two chemical elements and a complex 3D substrate on a substrate holder;
providing a gas to be ionized into the process chamber with a controlled pressure;
applying a voltage in pulse between the sputtering target and the complex 3D substrate; and
generating a magnetic field at a surface of the sputtering target inside the process chamber as required for High Power Impulse Magnetron Sputtering (HIPIMS).

28. The process of claim 27, wherein the at least two chemical elements are selected from the group consisting of transition metals, poor metals, metalloids, and polyatomic nonmetals.

29. The process of claim 27, wherein the at least two chemical elements are copper (Cu) and titanium dioxide (TiO₂).

30. The process of claim 29, wherein the at least two different chemical elements are present in a ratio of 40 at. % for copper (Cu) and 60 at. % for titanium oxide (TiO₂).

31. The process of claim 27, further comprising a step of:
controlling a distance between the sputtering target and the substrate to be coated in the process chamber.

32. The process of claim 27, wherein the distance between the sputtering target and the substrate to be coated is set at 10.5 cm.

33. The process of claim 27, wherein the gas is a mixture of an inert gas and a reactive gas.

34. The process of claim 27, wherein the gas is a mixture of Argon and Oxygen.

35. The process of claim 34, wherein the mixture of Argon and Oxygen is in a ratio of Flux_{O2}/Flux_{Ar}=0.05.

36. The process of claim 27, wherein the voltage is applied so that the pulse has a power per pulse in a range of 1000 W to 2000 W and has a duration in a range of 50 μs to 200 μs.

37. The process of claim 35, wherein the power per pulse is 1750 W and the pulse has duration of 100 μs.

38. The process of claim 27, further comprising
selecting process conditions including at least one of sputtering target composition, distance between the sputtering target and the substrate holder, gas or gas mixture, gas pressure, voltage in pulse, and magnetic field such that the film to be deposited will include the at least two chemical elements in multiple controlled oxidation states.

39. An apparatus for magnetically enhanced sputtering, comprising:
a process chamber including a sputtering target;
a substrate holder configured to hold a substrate to be coated;
a gas inlet inside the process chamber for providing a gas inside the process chamber; and
a power supply configured to apply a pulsed voltage between the sputtering target and the substrate to be coated and to generate a magnetic field, wherein
the sputtering target includes at least two different chemical elements.

40. The apparatus of claim 39, wherein, for the sputtering target, the at least two different chemical elements are selected from the group consisting of transition metals, poor metals, metalloids, and polyatomic nonmetals.

41. The apparatus of claim 39, wherein the at least two different chemical elements are copper (Cu) and titanium oxide (TiO₂).

42. The apparatus of claim 41, wherein the at least two different chemical elements are present in a ratio of 40 at. % for copper (Cu) and 60 at. % for titanium oxide (TiO₂).

43. The apparatus of claim 39, wherein, for the process chamber, the substrate holder is mounted with mounting means in the process chamber so that a distance between the sputtering target and the substrate to be coated can be controlled.

44. The apparatus of claim 43, wherein the distance between the sputtering target and the substrate to be coated is set at 10.5 cm.

45. The apparatus of claim 39, wherein the gas is a mixture of an inert gas and a reactive gas.

46. The apparatus of claim 45, wherein the gas is a mixture of Argon and Oxygen.

47. The apparatus of claim 46, wherein the mixture of Argon and Oxygen is in a ratio of Flux_{O2}/Flux_{Ar}=0.05.

48. The apparatus of claim 39, wherein a voltage is applied in pulse between the sputtering target and the substrate to be coated so that the pulse have a power per pulse in a range of 1000 W to 2000 W and have a duration in a range of 50 μs to 200 μs.

49. The apparatus of claim 48, wherein the power per pulse is 1750 W and the pulse has a duration of 100 μs.

50. An active film as prepared with the process of claim 27.

51. The active film of claim 50, wherein at least one of the at least two chemical element is in several oxidation states.

52. The active film of claim 50, wherein the active film is a bioactive surface.
